(19) United States
(12) Patent Application Publication
(10) Pub. No.: US 2025/0144100 A1
(43) Pub. Date: May 8, 2025

(54) PHARMACEUTICAL COMPOSITION COMPRISING IBRUTINIB

(71) Applicant: Synthon B.V., Nijmegen (NL)

(72) Inventors: Marta VIVANCOS MARTINEZ, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 18/717,130

(22) PCT Filed: Dec. 6, 2022
(86) PCT No.: PCT/EP2022/084622

§ 371 Date: Jun. 6, 2024

(30) Foreign Application Priority Data
Dec. 10, 2021 (EP) 21213878.8

(51) Int. Cl. A61K 31/519; A61K 9/20

(52) U.S. Cl. CPC A61K 31/519; A61K 9/2013; A61K 9/2095

(57) ABSTRACT

The present invention relates to a tablet composition comprising Ibrutinib and one ore more pharmaceutically acceptable excipients. The invention further relates to the use of said composition as a medicament, particularly in the treatment of lymphocytic leukaemia (CLL), mantle cell lymphoma (MCL), Waldenström's macroglobulinaemia (WM) and chronic graft versus host disease (cGVHD).

### BACKGROUND OF THE PRESENT INVENTION

The invention relates to a pharmaceutical composition comprising Ibrutinib, chemically 1-[(3R)-3-[4-amino-3-(4-phenoxyphenyl)-1H-pyrazolo[3,4-d]pyrimidin-1-yl]piperidin-1-yl]prop-2-en-1-one of formula (I):

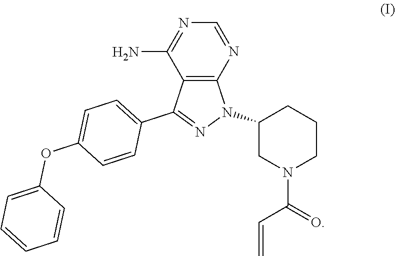 [SMILES: C=CC(=O)N1CCC[C@@H](n2nc(-c3ccc(Oc4ccccc4)cc3)c3c(N)ncnc32)C1]

Ibrutinib works by blocking an enzyme called Bruton's tyrosine kinase (Btk), which is mostly found in B lymphocytes. Btk promotes survival of B lymphocytes and their migration to the organs where these cells normally divide. By blocking Btk, Ibrutinib decreases survival and migration of B lymphocytes, thereby delaying the progression of the cancer.

Ibrutinib is marketed by Janssen/Pharmacyclics under the brand name Imbruvica® and is disclosed in WO2008039218. Ibrutinib is used for the treatment of chronic lymphocytic leukaemia (CLL) in previously untreated patients and in patients who have received at least one previous treatment. It is also used for the treatment of mantle cell lymphoma (MCL) in patients whose disease does not respond to or has come back after previous treatment and to treat Waldenström's macroglobulinaemia (WM), also known as lymphoplasmacytic lymphoma, in patients who have had previous treatment or who cannot have chemo immunotherapy. Most recently Ibrutinib was approved by United States Food and Drug Administration for the treatment of chronic graft versus host disease (cGVHD) after failure of one or more lines of systemic therapy.

Imbruvica® was initially only supplied as hard capsule in one strength: 140 mg. Patients take either 3 or 4 capsules once daily, depending on the disease to be treated. Later, the FDA approved a new tablet formulation of Imbruvica®. The tablet is available in the strengths 140, 280, 420 and 560 mg and thus reduces pill burden for patients.

The object of the present invention is to provide stable pharmaceutical compositions of Ibrutinib suitable for production on commercial scale and bioequivalent to Imbruvica®.

### BRIEF DESCRIPTION OF THE PRESENT INVENTION

The present invention provides a tablet composition comprising Ibrutinib. The tablet composition according to the present invention comprises lubricant sodium stearyl fumarate and one or more pharmaceutically acceptable excipients.

It also provides a process for preparing the tablet composition comprising a granulation step.

Said pharmaceutical composition may be used as a medicament, particularly in the treatment of chronic lymphocytic leukaemia (CLL), mantle cell lymphoma (MCL), Waldenström's macroglobulinaemia (WM) and chronic graft versus host disease (cGVHD).

### BRIEF DESCRIPTION OF THE DRAWINGS

FIG. 1 shows dissolution profile of formulations of Example 1 (1.A and 1.B) in comparison to Imbruvica®

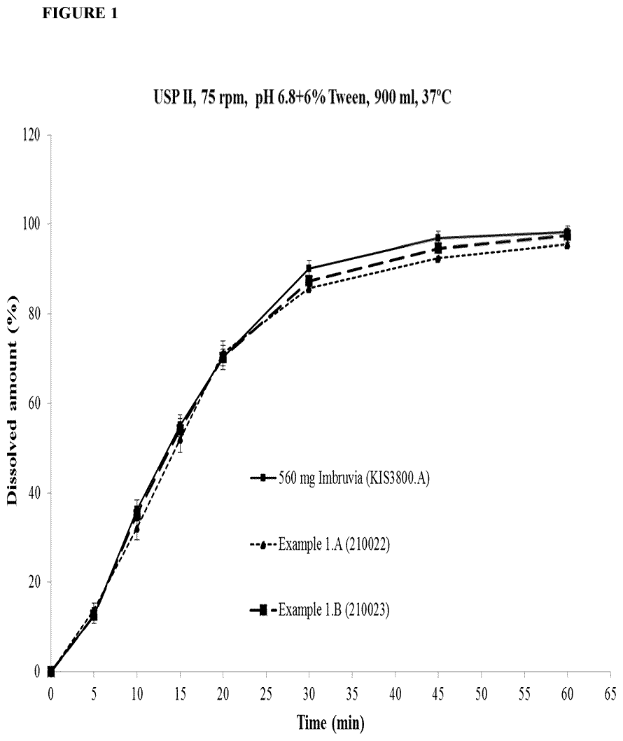

FIG. 2 shows dissolution profile of formulation of Example 2 in comparison to Imbruvica®

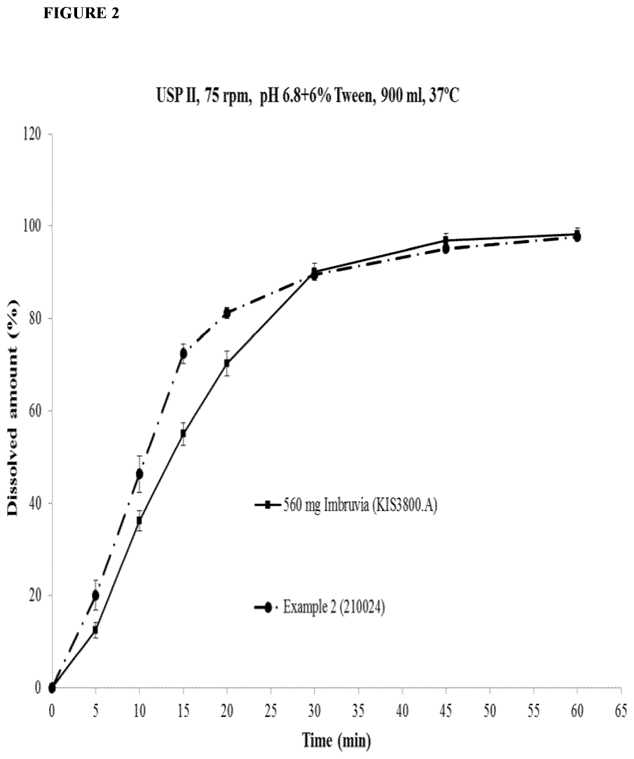

FIG. 3 shows dissolution profile of formulation of Example 3 in comparison to Imbruvica®

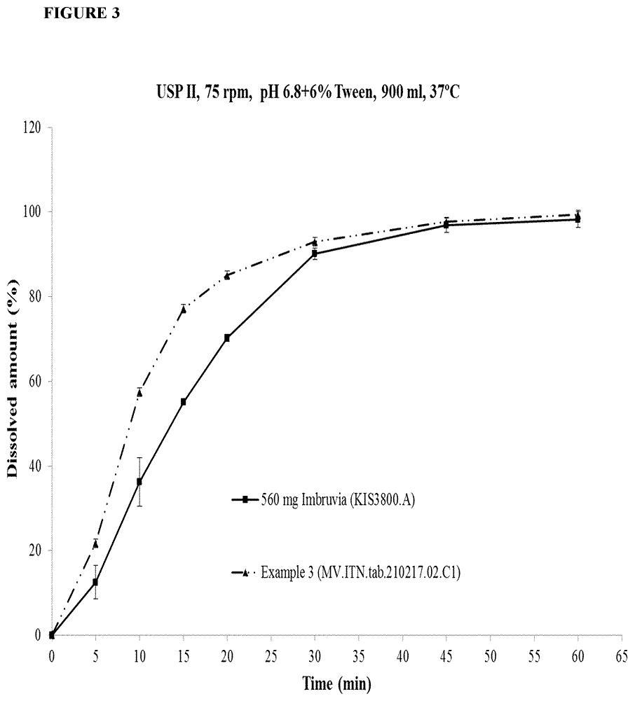

### DETAILED DESCRIPTION OF THE PRESENT INVENTION

Imbruvica® tablets are available in the strengths 140, 280, 420 and 560 mg and thus reduce pill burden for patients. The development of a robust tablet formulation for a high dose active pharmaceutical ingredient is challenging as the tablet cannot be too large for the patient to take. Ibrutinib is a BCS class II compound, exhibiting low solubility and high permeability. Therefore, the excipients to be used in the formulation need be carefully selected so the final dosage form performs as desired.

In pharmaceutical tablet manufacturing, the tablet ejection force is a common parameter monitored during the compression run. Ejection force is the force required to the push the tablet out of the die. Pharmaceutical powders can exhibit markedly different ejection forces when compressed at a pressure typical of tablet manufacturing. Tablet ejection force also often increases with increasing tableting speed. High ejection force is the sign that the process or product is susceptible to such risks as punch sticking or tablet defects.

Lubricants are routinely added to pharmaceutical formulations to reduce ejection force during tableting and to avoid accumulation of powder on tablet tooling. Therefore, the choice of the proper lubricant and its amount is very important factor for the large scale manufacturing at a high speed. Magnesium stearate is a frequently used lubricant in tablet manufacturing. Typical excipient levels used in immediate-release formulations range between 0.25-5% w/w. Commercial Ibrutinib tablet Imbruvica® comprises 0.5% w/w of magnesium stearate. It was discovered, that the tablets comprising Ibrutinib prepared with 0.5% of magnesium stearate tend to have sticking issues. It is known that magnesium stearate can compromise drug release, as it acts as water repellent through the formation of a hydrophobic film around drug particles. Therefore, the use of higher amount of this lubricant in the Ibrutinib composition could lead to lower bioavailability of Ibrutinib.

It was surprisingly found by the present inventors that the high load Ibrutinib tablet composition exhibiting a similar dissolution profile as Imbruvica® but without tendency to stick can be prepared with the use of sodium stearyl fumarate as lubricant. Even when sodium stearyl fumarate is present at the higher amount, the tablet composition exhibits similar dissolution profile to Imbruvica®.

The present invention relates to a pharmaceutical tablet composition comprising from 60% to 85% of Ibrutinib, sodium stearyl fumarate and one or more pharmaceutically acceptable excipients, wherein the amount of sodium stearyl fumarate in the composition is from about 1.5% to about 3%, preferably from 1.8% to 2.2% by weight relative to the total weight of the tablet. In the most preferred embodiment the amount of sodium stearyl fumarate in the tablet composition is 2% by weight relative to the total weight of the tablet. Sodium stearyl fumarate is preferably added as extragranular component.

The pharmaceutical tablet composition of the invention comprises a therapeutically effective dose of Ibrutinib. Ibrutinib used in the tablet composition has a particle size distribution D90 from 3 to 100 μm, preferably from 4 to 90 μm, more preferably from 5 to 80um measured by laser diffraction analysis. The D90 value of the particle size distribution is defined as the particle diameter at which 90% by volume of the particles have a smaller diameter than the diameter which corresponds to the D90 value measured by laser diffraction analysis. Specifically, a Malvern Instruments Mastersizer was used to determine the particle size distribution.

In a preferred embodiment of the present invention Ibrutinib is present in an amount of 70% by weight based on the total composition weight. Ibrutinib is preferably present intragranularly.

The tablet compositions according to the present invention comprise, besides Ibrutinib and sodium stearyl fumarate, one or more pharmaceutically acceptable excipients. The excipients to be used in accordance with the present invention are well-known and are those excipients which are conventionally used by the person skilled in the art. The pharmaceutically acceptable excipients are chosen from one or more fillers, binders, disintegrants, glidants and surfactants.

Fillers are used to increase the bulk volume of a tablet or capsule. By combining a filler with the active pharmaceutical ingredient, the final product is given adequate weight and size to assist in production and handling. The pharmaceutical composition of the present invention preferably comprises at least one filler.

The fillers to be used in accordance with the present invention may be any filler known to a person of ordinary skill in the art. Particularly, the filler to be used in accordance with the present invention is an inorganic filler, polysaccharide, mono-or disaccharide or sugar alcohol. Lactose and microcrystalline cellulose are particularly preferred fillers.

In one embodiment of the present invention, the filler is added as intragranular component. In another embodiment, the filler is added partially to the intragranular phase and partially to the extragranular phase.

The filler to be used in accordance with the present invention may contain lactose, microcrystalline cellulose, or mixture of lactose and microcrystalline cellulose.

Fillers are preferably used in an amount of from 10% to 30%, more preferably of from 15% to 25% by weight based on the total weight of the tablet composition.

The pharmaceutical composition of the present invention may also comprise one or more binders. Binders ensure that tablets and granules can be formed having the desired or required mechanical strength.

The binder to be used in accordance with the present invention may be any binder known to a person of ordinary skill in the art. Suitable binders are selected from the group consisting of sodium carboxymethylcellulose, polyvinyl pyrrolidone (PVP), copovidone, polyvinyl pyrrolidone-vinyl acetate (PVP/VA) copolymer, hydroxypropylcellulose (HPC), hydroxypropyl methylcellulose (HPMC), ethyl cellulose, pregelatinized starch, starch polyethylene glycol (PEG), gelatine, glucose and alginic acid. HPMC is particularly preferred binder.

The binder is added to the intragranular phase.

The tablet composition according to the present invention comprises from 0% to 5% by weight of one or more binders, relative to the total weight of the tablet composition. More preferably, the composition comprises 0% to 3% by weight of a binder, even more preferably from 0% to 1% by weight of binder, all relative to the total weight of the tablet composition.

The pharmaceutical composition of the present invention may also comprise one or more disintegrants. Disintegrants are added to a tablet or capsule composition to promote the breakup of the tablet/capsule into smaller fragments in an aqueous environment, thereby increasing the available surface area and promoting a more rapid release of the active pharmaceutical ingredient. The disintegrant to be used in accordance with the present invention may be any disintegrant known to a person of ordinary skill in the art. Suitable disintegrants to be used in accordance with the present invention are selected from the group consisting of croscarmellose sodium, crospovidone, low-substituted hydroxypropylcellulose (HPC) or sodium starch glycolate. Croscarmellose sodium is a particularly preferred disintegrant.

In one embodiment of the present invention, the disintegrant is added as to the intragranular phase. In another embodiment, the disintegrant is added partially to the intragranular phase and partially to the extragranular phase.

Disintegrants are preferably used in an amount of from 2% to 15% by weight, more preferably of from 4% to 10% by weight based on the total weight of the composition.

The pharmaceutical composition of the present invention may also comprise one or more glidants. Glidants enhance product flow by reducing interparticulate friction. The glidant to be used in accordance with the present invention may be any glidant known to a person of ordinary skill in the art. Colloidal silicon dioxide is a particularly preferred glidant.

In one embodiment of the present invention, the glidant is added as intragranular component. In another embodiment, the glidant is added to the extragranular phase.

Glidants are preferably used in an amount of from 0.25% to 1% by weight, more preferably of from 0.4% to 0.7% by weight based on the total weight of the composition.

The tablet composition of the present invention can further comprise surfactant. Surfactants can have multiple roles in the pharmaceutical preparations. They can modulate the solubility and bioavailability of APIs and increase the stability of active ingredients in the dosage forms The surfactant to be used in accordance with the present invention may be any surfactant known to a person of ordinary skill in the art. Sodium lauryl sulfate is particularly preferred surfactant to be used in tablet composition of the present invention.

The surfactant is added into the intragranular phase.

The tablet composition comprises preferably 0.5 to 3% by weigh of one or more surfactants, relative to the total weight of the tablet. More preferably the tablet composition comprises 1 to 2% by weight of surfactant, relative to the total weight of the tablet.

The tablet composition according to the present invention preferably comprises 60% to 85% by weight of Ibrutinib, 1.5% to 3% by weight of sodium stearyl fumarate, 10 to 30% by weight of one or more fillers, 0 to 5% by weight of one or more binders, 2 to 15% by weight of one or more disintegrants, 0.25 to 1.0% by weight of one or more glidants and 0.5% to 3% by weight of one or more surfactants, all relative to the total tablet weight.

The tablets may be optionally further coated by a film-coat. The coating serves generally for cosmetic purposes. The coating material typically has no influence on the release rate, except of an inherent short initial delay in dissolution due to the time necessary to dissolve the coat. The coating may be selected from amongst one or more of those suitable coating materials known in the art.

The coating may be performed by applying one or more film forming polymers, with or without other pharmaceutically inert excipients, as a solution/suspension. Coating is done using any conventional coating technique known in the art, such as spray coating in a conventional coating pan or fluidized bed processor; or dip coating.

The tablet composition according to the present invention is packaged in primary packaging material, e.g. blisters and bottles.

The pharmaceutical composition of the present invention exhibits excellent long term stability. Moreover, the pharmaceutical composition of the present invention is very suitable for production on commercial scale making use of equipment and techniques commonly used in industry.

The present invention further provides a process to prepare a tablet composition comprising Ibrutinib, sodium stearyl fumarate one or more pharmaceutically acceptable excipients comprising a granulation step. The granulation processes applied are simple and cost effective and include a standard wet or dry granulation technique.

The wet granulation process is performed with a granulation solvent selected from the group consisting of water, acetone, ethanol, isopropanol or a mixture thereof.

Preferably, the process to prepare the tablet composition of the present invention comprises a wet granulation step.

The tablet prepared by applying the step of wet granulation comprises, besides Ibrutinib and sodium stearyl fumarate, one or more pharmaceutically acceptable binders, fillers, disintegrants, glidants or surfactants. Preferably, the tablet prepared by using the step of wet granulation comprises Ibrutinib, sodium stearyl fumarate, lactose, microcrystalline cellulose, croscarmellose sodium, silicon dioxide and sodium lauryl sulfate. Optionally hydroxypropyl methylcellulose may be present as well.

The tablet composition in accordance with the present invention may be used as a medicament. The composition typically may be used in the treatment of chronic lymphocytic leukaemia (CLL), mantle cell lymphoma (MCL), Waldenström's macroglobulinaemia (WM) and chronic graft versus host disease (cGVHD).

The following examples are intended to illustrate the scope of the present invention but not to limit it thereto.

### EXAMPLES

### Comparative Example 1

The tablets comprising Ibrutinib have been prepared with the compositions as given in table 1.

TABLE 1

| | Ibrutinib tablet composition | | | | | | | |
|---|---|---|---|---|---|---|---|---|
| | 1 | | 2 | | 3 | | 4 | |
| | mg/ | | mg/ | | mg/ | | mg/ | |
| Component | tablet | % | tablet | % | tablet | % | tablet | % |

Intragranular components

| | | | | | | | | |
|---|---|---|---|---|---|---|---|---|
| Ibrutinib | 560.0 | 70.0 | 560.0 | 70.0 | 560.0 | 70.0 | 560.0 | 70.0 |
| Lactose monohydrate | 112.0 | 14.0 | 112.0 | 14.0 | 112.0 | 14.0 | 112.0 | 14.0 |
| Croscarmellose sodium | 40.0 | 5.0 | 40.0 | 5.0 | 40.0 | 5.0 | 40.0 | 5.0 |
| Sodium lauryl sulfate | 8.0 | 1.0 | 8.0 | 1.0 | 8.0 | 1.0 | 8.0 | 1.0 |

Extragranular components

| | | | | | | | | |
|---|---|---|---|---|---|---|---|---|
| Microcrystalline cellulose | 56.0 | 7.0 | 52.0 | 6.5 | 48.0 | 6.0 | 44.0 | 5.5 |
| Croscarmellose sodium | 16.0 | 2.0 | 16.0 | 2.0 | 16.0 | 2.0 | 16.0 | 2.0 |
| Colloidal silicon dioxide | 4.0 | 0.5 | 4.0 | 0.5 | 4.0 | 0.5 | 4.0 | 0.5 |
| Magnesium stearate | 4.0 | 0.5 | — | — | — | — | — | — |
| Sodium stearyl fumarate | — | — | 8.0 | 1.0 | 12.0 | 1.5 | 16.0 | 2.0 |
| Total core tablet weight | 800.0 | 100.0 | 800.0 | 100.0 | 800.0 | 100.0 | 800.0 | 100.0 |

The tablets were prepared by using the process of wet granulation. The granulate comprising Ibrutinib, lactose, intragranular portion of croscarmellose sodium and sodium lauryl sulfate was mixed with the extragranular components and compressed using a rotating tablet press using appropriate punches in the MODUL™ P Tablet Press.

TABLE 2

| | 1 | 2 | 3 | 4 |
|---|---|---|---|---|
| Tablet appearance | Sticking | Correct | Correct | Correct |
| Ejection force (KN) | 0.91 | 0.73 | 0.50 | 0.35 |

Formulation made with 1%, 1.5% and 2% of sodium stearyl fumarate as lubricant showed a correct tablet appearance. The higher the amount of sodium stearyl fumarate, the lower the ejection force of the tablet was obtained.

### Example 1

Tablets were prepared using Ibrutinib having following particle size distribution:

| | Example 1.A | Example 1.B |
|---|---|---|
| Ibrutinib D90 | 73.7 μm | 8.0 μm |

The film-coated tablets comprising Ibrutinib with two different particle size distribution were prepared by the process of wet granulation and have the composition as given in table 3.

TABLE 3

Ibrutinib tablet composition

| | | |
|---|---|---|
| Component | mg/tablet | % |

Intragranular components

| | | |
|---|---|---|
| Ibrutinib | 560.0 | 70.00 |
| Lactose monohydrate | 112.0 | 14.00 |
| Hydroxypropyl methylcellulose | 8.0 | 1.00 |
| Croscarmellose sodium | 40.0 | 5.00 |
| Sodium lauryl sulfate | 8.0 | 1.00 |

Extragranular components

| | | |
|---|---|---|
| Microcrystalline cellulose | 36.0 | 4.50 |
| Croscarmellose sodium | 16.0 | 2.00 |
| Colloidal silicon dioxide | 4.0 | 0.50 |
| Sodium stearyl fumarate | 16.0 | 2.00 |
| Total core tablet weight | 800.0 | 100.0 |

Sodium lauryl sulfate and hydroxypropyl methylcellulose were dissolved in water. Ibrutinib, lactose monohydrate and intragranular portion of croscarmellose sodium were sieved through a 1.5 mm mesh size and mixed by using a diffusion mixer. The obtained blend was transferred to a high shear mixer and the solution comprising sodium lauryl sulfate and hydroxypropyl methylcellulose was added. A granulation was carried out. The wet granules were sieved through 3 mm mesh and dried in a fluid bed drier at 60-70° C. The dry granules were sieved using a screening mill with a 1.1 mm mesh size and transferred into an appropriate bin blender. Microcrystalline cellulose, colloidal silicon dioxide and extragranular portion of sodium croscarmellose were sieved through 1.1 mm mesh size and blended with the sieved granules for 10 min at 20 rpm. Sodium stearyl fumarate was sieved through 0.5 mm mesh sieve and mixed with the blend in a bin blender for 3 min at 20 rpm. The homogeneous blend was compressed on a tablet press. The tablets were coated with a water suspension of Opadry® II yellow until 3% of weight gain.

### Example 2

The film-coated tablets comprising Ibrutinib prepared by the process of wet granulation and have the composition as given in table 4.

TABLE 4

Ibrutinib tablet composition

| | | |
|---|---|---|
| Component | mg/tablet | % |

Intragranular components

| | | |
|---|---|---|
| Ibrutinib | 560.0 | 70.00 |
| Lactose monohy drate | 112.0 | 14.00 |
| Hydroxypropyl methylcellulose | 2.1 | 0.26 |
| Croscarmellose sodium | 40.0 | 5.00 |
| Sodium lauryl sulfate | 8.0 | 1.00 |

Extragranular components

| | | |
|---|---|---|
| Microcrystalline cellulose | 41.9 | 5.24 |
| Croscarmellose sodium | 16.0 | 2.00 |
| Colloidal silicon dioxide | 4.0 | 0.50 |
| Sodium stearyl fumarate | 16.0 | 2.00 |
| Total core tablet weight | 800.0 | 100.00 |

Sodium lauryl sulfate and hydroxypropyl methylcellulose were dissolved in water. Ibrutinib, lactose monohydrate and intragranular portion of croscarmellose sodium were sieved through a 1.5 mm mesh size and mixed by using a diffusion mixer. The obtained blend was transferred to a high shear mixer and the solution comprising sodium lauryl sulfate and hydroxypropyl methylcellulose was added. A granulation was carried out. The wet granules were sieved through 3 mm mesh and dried in a fluid bed drier at 60-70° C. The dry granules were sieved using a screening mill with a 1.1 mm mesh size and transferred into an appropriate bin blender. Microcrystalline cellulose, colloidal silicon dioxide and extra-granular portion of sodium croscarmellose were sieved through 1.1 mm mesh size and blended with the sieved granules for 10 min at 20 rpm. Sodium stearyl fumarate was sieved through 0.5 mm mesh sieve and mixed with the blend in a bin blender for 3 min at 20 rpm.

The homogeneous blend was compressed on a tablet press. The tablets were coated with a water suspension of Opadry® II yellow until 3% of weight gain.

### Example 3

The film-coated tablets comprising Ibrutinib prepared by the process of wet granulation and have the composition as given in table 5.

TABLE 5

Ibrutinib tablet composition

| | | |
|---|---|---|
| Component | mg/tablet | % |

Intragranular components

| | | |
|---|---|---|
| Ibrutinib | 560.0 | 70.00 |
| Lactose monohydrate | 112.0 | 14.00 |
| Croscarmellose sodium | 40.0 | 5.00 |
| Sodium lauryl sulfate | 16.0 | 2.00 |

Extragranular components

| | | |
|---|---|---|
| Microcrystalline cellulose | 36.0 | 4.50 |
| Croscarmellose sodium | 16.0 | 2.00 |
| Colloidal silicon dioxide | 4.0 | 0.50 |
| Sodium stearyl fumarate | 16.0 | 2.00 |
| Total core tablet weight | 800.0 | 100.00 |

Sodium lauryl sulfate was dissolved in water. Ibrutinib, lactose monohydrate and intragranular portion of croscarmellose sodium were sieved through a 1.5 mm mesh size and mixed by using a diffusion mixer. The obtained blend was transferred to a high shear mixer and the solution comprising sodium lauryl sulfate was added. A granulation was carried out. The wet granules were sieved through 3 mm mesh and dried in a fluid bed drier at 60-70° C. The dry granules were sieved using a screening mill with a 1.1 mm mesh size and transferred into an appropriate bin blender. Microcrystalline cellulose, colloidal silicon dioxide and extragranular portion of sodium croscarmellose were sieved through 1.1 mm mesh size and blended with the sieved granules for 10 min at 20 rpm. Sodium stearyl fumarate was sieved through 0.5 mm mesh sieve and mixed with the blend in a bin blender for 3 min at 20 rpm.

The homogeneous blend was compressed on a tablet press. The tablets were coated with a water suspension of Opadry® II yellow until 3% of weight gain.

1. A high load tablet comprising from 60 to 85% by weight of Ibrutinib relative to the total weight of the tablet, from 1.5% to 3% by weight of sodium stearyl fumarate relative to the total weight of the tablet, and one or more pharmaceutically acceptable excipients.

2. The tablet according to claim 1, wherein the amount of sodium stearyl fumarate is from 1.8% to 2.2% by weight relative to the total weight of the tablet.

3. The tablet according to claim 1, wherein the Ibrutinib has a particle size distribution D90 is less than 100 micrometers when measured by laser diffraction analysis.

4. The tablet composition according to claim 1, wherein the pharmaceutically acceptable excipients are chosen from one or more fillers, binders, disintegrants, glidants and surfactants.

5. The tablet according to claim 1, comprising:
a) Ibrutinib in an amount from 60 to 85% by weight;
b) Sodium stearyl fumarate in an amount from 1.5 to 3% by weight;
c) One or more fillers in an amount from 10 to 30% by weight;
d) One or more binders in an amount from 0 to 5% by weight;
e) One or more disintegrants in an amount from 2 to 15% by weight;
f) One or more glidants in an amount from 0.25 to 1.0% by weight; and
g) One or more surfactants in an amount from 0.5 to 3% by weight;
all relative to the total weight of the tablet.

6. The tablet according to claim 1 prepared by wet or dry granulation.

7. The tablet according to claim 6 prepared by wet granulation.

8. The tablet according to claim 6, wherein the Ibrutinib is contained intragranularly in said tablet.

9. The tablet according to claim 6, wherein said sodium stearyl fumarate is contained extragranularly in said tablet.

10. The tablet according to claim 5, wherein said tablet has an intragranular component and an extragranular component; and wherein said Ibrutinib is present in the intragranular component and said sodium stearyl fumarate is present in the extragranular component.

11. The tablet according to claim 10, wherein the amount of sodium stearyl fumarate is from 1.8% to 2.2% by weight relative to the total weight of the tablet.
